(19) United States
(12) Patent Application Publication
(10) Pub. No.: US 2010/0041915 A1
(43) Pub. Date: Feb. 18, 2010

(54) METHOD FOR PRODUCING ISOCYANATES

(75) Inventors: Andreas Woelfert, Bad Rappenau (DE); Carsten Knoesche, Niederkirchen (DE); Torsten Mattke, Freinsheim (DE); Eckhard Stroefer, Mannheim (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/521,878

(22) PCT Filed: Dec. 12, 2007
(86) PCT No.: PCT/EP2007/063771

§ 371 Date: Jul. 1, 2009

(30) Foreign Application Priority Data
Jan. 17, 2007 (EP) 07100644.9

(51) Int. Cl. C07C 263/10

(52) U.S. Cl. 560/347

(57) ABSTRACT

The present invention relates to a process for preparing diisocyanates from diamines and phosgene in the gas phase.

The present invention relates to a process for preparing diisocyanates from diamines and phosgene in the gas phase.

EP 570799, example 1, describes the work-up of a reaction mixture obtained by gas-phase phosgenation by means of a scrubbing tower through which water trickles to separate off phosgene and hydrogen chloride.

Such a work-up destroys excess phosgene and hydrogen chloride gas so that they can no longer be beneficially used in the reaction.

EP 593334 B1 and EP 699657 B1 disclose the possibility of utilizing or destroying phosgene or hydrogen chloride gas, but without going into the specific problems associated with recirculated phosgene.

EP 749 958 B1 paragraph [0018] and EP 1078918 B1 paragraph [0018] mention the possibility of recovering excess phosgene after a gas-phase phosgenation of triamines and reusing recovered hydrogen chloride gas in the phosgene synthesis.

Here too, no detailed description of the recirculated phosgene is given.

U.S. Pat. No. 4,581,174 described the continuous preparation of organic monoisocyanates and/or polyisocyanates by phosgenation of the primary amine in a mixing circuit with partial recirculation of the isocyanate-comprising reaction mixture, with the proportion of HCl in the recirculated mixture being less than 0.5%. Here too, the continuous recirculation of the isocyanate to the reaction zone with free amine promotes the formation of urea. The precipitated urea endangers stable operation of the process.

GB 737 442 describes the recovery of phosgene from the isocyanate synthesis. The recovered phosgene has an HCl content of from 0.5 to 0.7%.

DE 10261191 A1 and WO 2004/58689 describe phosgenations in which the HCl content of the phosgene-comprising feed stream is less than 0.4 or more than 0.8% by weight.

These documents do not make a distinction between the problems involved in gas-phase phosgenation and those involved in liquid-phase phosgenation and preferably relate only to liquid-phase phosgenation.

A disadvantage of all these processes is that the chlorine content of the phosgene or during the course of the phosgenation is disregarded.

The international patent application having the number PCT/EP2006/064850 and the filing date Jul. 31, 2006 describes a gas-phase phosgenation process in which the content of hydrogen chloride is supposed to remain below a particular threshold.

WO 04/56758 mentions chlorine as a constituent of phosgene in a broad list of secondary components, but does not disclose any teachings about the specific problems of the chlorine content in a gas-phase phosgenation.

U.S. Pat. No. 3,331,873 discloses a general method of separating chlorine from phosgene by means of activated carbon to a content of less than 25 ppm.

In the process disclosed, no reference is made to the specific problems in phosgenations and in particular in gas-phase phosgenations.

WO 01/00569 describes the effect of the content of bromine and bromine-comprising compounds on the color number in liquid-phase phosgenations under a pressure of up to 100 bar and a temperature of 0-130° C.

In the process disclosed, no reference is made to the specific problems in gas-phase phosgenations.

The gas-phase phosgenation is usually carried out at temperatures of from 200 to 600° C. Due to these high temperatures, the design of the process has to meet particular requirements in order to achieve long-term operation of the process without leakages resulting from increased stresses on materials and in particular the reactor wall in the high-temperature range.

The high temperatures in combination with the corrosive reaction media result in specific demands on the process and the materials used. For example, it is known that at high temperatures (at or above about 400° C.) phosgene dissociates autocatalytically into molecular chlorine (Cl₂) and carbon monoxide (CO). At high temperatures, chlorine leads to embrittlement of materials, presumably by incorporation into the material. Materials and in particular reactor walls which have been embrittled in this way can be stressed in the event of, for example, unavoidable vibrations in the production plants and rupture or break, so that the probability of leakage is increased. In addition, chlorine can react exothermically with unalloyed steels at above 170° C. in a chlorine-iron fire. This is industrially problematical, especially in the handling of the very toxic phosgene.

It was therefore an object of the invention to provide a process which allows the reaction of diamines with phosgene in the gas-phase to form the corresponding diisocyanates and hydrogen chloride (HCl) to be carried out in such a way that embrittlement of materials can be reduced.

The object has been achieved by a process for preparing diisocyanates by reacting the corresponding diamines with a stoichiometric excess of phosgene in at least one reaction zone,

wherein the reaction conditions are selected so that at least the reaction components diamine, diisocyanate and phosgene are gaseous under these conditions and at least one diamine-comprising gas stream and at least one phosgene-comprising gas stream are fed into the reaction zone,
with the mass fraction of chlorine in the phosgene-comprising stream before mixing with the amine-comprising stream being less than 1000 ppm by weight and/or the mass fraction of bromine in the phosgene comprising stream being less than 50 ppm by weight.

In the gas-phase phosgenation, efforts should be made, according to the invention, to ensure that the compounds occurring during the course of the reaction, i.e. starting materials (diamine and phosgene), intermediates (in particular the monocarbamoyl and dicarbamoyl chlorides formed as intermediates), end products (diisocyanate) and any inert compounds introduced, remain in the gas phase under the reaction conditions. Should these or other components deposit from the gas phase onto, for example, the reactor wall or other components of the apparatus, the heat transfer or the flow through the components affected can be undesirably altered by these deposits. This applies in particular to amine hydrochlorides which are formed from free amino groups and hydrogen chloride (HCl), since the resulting amine hydrochlorides precipitate easily and are difficult to vaporize again.

In a preferred embodiment, it is possible, in addition to the reduced chlorine content according to the invention, for the mass fraction of hydrogen chloride in the phosgene-comprising stream after any mixing with fresh phosgene and before mixing with the amine-comprising stream to be less than 15% by weight, preferably less than 10% by weight, particularly preferably less than 5% by weight.

The result of this is that the formation of amine hydrochlorides can be reduced decisively, so that the risk of deposit formation in the reactor is reduced.

Due to the low content according to the invention of chlorine, i.e. in this text molecular chlorine (Cl₂), in the phosgene fed into the reaction, it is possible to keep the total chlorine content during the course of the conversion of the diamine into the corresponding diisocyanate as low as possible despite the chlorine formed by dissociation of phosgene during the course of the reaction, so that the risk of embrittlement of materials and/or chlorine-iron fires can be reduced or even ruled out.

According to the invention, the chlorine content of the phosgene-comprising stream after any mixing with fresh phosgene and before mixing with the amine-comprising stream is less than 1000 ppm by weight, preferably less than 500 ppm by weight, particularly preferably less than 250 ppm by weight, very particularly preferably less than 100 ppm by weight, in particular less than 50 ppm by weight and especially less than 25 ppm by weight.

In addition or as an alternative thereto, the content of bromine or iodine or mixtures thereof in molecular or bound form in the phosgene-comprising stream after any mixing with fresh phosgene and before mixing with the amine-comprising stream is less than 50 ppm by weight, preferably less than 40 ppm by weight, 35 ppm by weight, 30 ppm by weight or 25 ppm by weight or less, in particular 10 ppm by weight or less and especially 5 ppm by weight, 3 ppm by weight, 2 ppm by weight or 1 ppm by weight or less.

For the purposes of the present text, bromine or iodine in molecular form are molecules which consist only of bromine or iodine atoms. The expression bromine or iodine in bound form refers to molecules which comprise not only bromine or iodine but also further atoms which are in each case different from the atoms mentioned. Unless indicated otherwise, the term “bromine” refers to molecular bromine (Br₂) in the present text.

In the process of the invention, the reaction of phosgene with diamine occurs in the gas phase.

Diisocyanates which can be prepared by the process of the invention can be aromatic, cycloaliphatic or aliphatic diisocyanates.

Cycloaliphatic isocyanates are ones which comprise at least one cycloaliphatic ring system.

Aliphatic isocyanates are ones which have exclusively isocyanate groups which are bound to straight or branched chains.

Aromatic isocyanates are ones which have at least one isocyanate group bound to at least one aromatic ring system.

For the purposes of the present patent application, the expression (cyclo)aliphatic isocyanates is used as an abbreviation for cycloaliphatic and/or aliphatic isocyanates.

Examples of aromatic diisocyanates are preferably those having 6-20 carbon atoms, for example monomeric methylenedi(phenyl isocyanate) (MDI), tolylene 2,4- and/or 2,6-diisocyanate (TDI) and naphthyl diisocyanate (NDI).

Diisocyanates are preferably (cyclo)aliphatic diisocyanates, particularly preferably (cyclo)aliphatic diisocyanates having from 4 to 20 carbon atoms.

Examples of customary diisocyanates are aliphatic diisocyanates such as tetramethylene diisocyanate, pentamethylene diisocyanate (1,5-diisocyanatopentane), 2-methyl-1,5-diisocyanatopentane, hexamethylene diisocyanate (1,6-diisocyanatohexane), octamethylene 1,8-diisocyanate, decamethylene 1,10-diisocyanate, dodecamethylene 1,12-diisocyanate, tetradecamethylene 1,14-diisocyanate, derivatives of lysine diisocyanate, tetramethylxylylene diisocyanate (TMXDI), trimethylhexane diisocyanate or tetramethylhexane diisocyanate and also 3 (or 4), 8 (or 9)-bis(isocyanatomethyl)tricyclo[5.2.1.0^{2.6}]decane isomer mixtures and also cycloaliphatic diisocyanates such as 1,4-, 1,3- or 1,2-diisocyanatocyclohexane, 4,4′- or 2,4′-di(isocyanatocyclohexyl)-methane, 1-isocyanato-3,3,5-trimethyl-5-(isocyanatomethyl)cyclohexane (isophorone diisocyanate), 1,3- or 1,4-bis(isocyanatomethyl)cyclohexane, 2,4- or 2,6-diisocyanato-1-methylcyclohexane.

Preference is given to 1,6-diisocyanatohexane, 1-isocyanato-3,3,5-trimethyl-5-(isocyanatomethyl)cyclohexane, 4,4′-di(isocyanatocyclohexyl)methane and tolylene diisocyanate isomer mixtures. Particular preference is given to 1,6-diisocyanatohexane, 1-isocyanato-3,3,5-trimethyl-5-(isocyanatomethyl)cyclohexane and 4,4′-di(isocyanatocyclohexyl)methane.

In the process of the invention, amines which can preferably be brought into the gas phase without significant decomposition can be used for the reaction to form the corresponding diisocyanates. Particularly suitable amines here are amines, in particular diamines, based on aliphatic or cycloaliphatic hydrocarbons having from 2 to 18 carbon atoms. Examples are 1,6-diaminohexane, 1-amino-3,3,5-trimethyl-5-aminomethylcyclohexane (IPDA) and 4,4′-diaminodicyclohexylmethane. Preference is given to using 1,6-diaminohexane (HDA).

It is likewise possible to use aromatic amines which can preferably be brought into the gas phase without decomposition for the process of the invention. Examples of preferred aromatic amines are tolylenediamine (TDA), as 2,4 or 2,6 isomer or as a mixture thereof, diaminobenzene, napthylenediamine (NDA) and 2,4′- or 4,4′-methylenedi(phenylamine) (MDA) or isomer mixtures thereof. Among these, preference is given to the diamines, particularly preferably 2,4- and/or 2,6-TDA.

The starting materials or else only one of them can be introduced together with an inert medium into the reaction space.

An additional inert medium can be introduced in the process of the invention. The Inert medium is a medium which at the reaction temperature is present in gaseous form in the reaction space and does not react with the compounds occurring during the course of the reaction. The inert medium is generally mixed with amine and/or phosgene before the reaction, but can also be fed in separately from the starting material streams. For example, it is possible to use nitrogen, noble gases such as helium or argon or aromatics such as chlorobenzene, dichlorobenzene, xylene, carbon dioxide or carbon monoxide. Preference is given to using nitrogen and/or chlorobenzene as inert medium.

In general, the inert medium is used in such an amount that the gas volume ratio of inert medium to amine or to phosgene is from >0.0001 to 30, preferably from >0.01 to 15, particularly preferably from >0.1 to 5.

The inert medium is preferably introduced into the reaction space together with the diamine.

The introduction of phosgene into the reaction space via the phosgene-comprising stream can also be effected by feeding in a plurality of phosgene-comprising substreams instead of a single phosgene-comprising stream. In such a case, the phosgene-comprising substreams are added up to a total of phosgene-comprising total stream and the mass fraction of chlorine in the phosgene-comprising total stream is obtained from the mass fractions of chlorine in the individual phosgene-comprising substreams under the assumption of retention of molecules without reaction. In this case, the value of the mass fraction of chlorine calculated in this way is used in the conceptual total phosgene stream.

An analogous situation applies to the bromine content.

Such substreams can be introduced in the following way:
Various phosgene-comprising substreams, for example recirculated phosgene and fresh phosgene, can be combined to form a phosgene-comprising total stream before introduction and be fed into the reaction space.
A plurality of substreams, which can in each case be recirculated phosgene, fresh phosgene or mixtures thereof, can be fed into the reaction space at the same place, for example via a plurality of nozzles which are arranged in parallel around a central nozzle, as is described, for example, in EP 1449826 A1, by introduction through an annular gap, as is described, for example, in the international patent application having the number PCT/EP2006/065593 and the filing date Aug. 23, 2006, or by multiple injection into an annular space for mixing before this stream is mixed with an amine-comprising stream introduced via a central nozzle.
A plurality of substreams, which can in each case be recirculated phosgene, fresh phosgene or mixtures thereof, can be introduced at various places in the reaction space so that further phosgene is introduced during the course of the reaction.

The term “fresh phosgene” thus refers to a phosgene-comprising stream which has not been recirculated from a phosgenation process and has not gone through a reaction stage involving a reaction of phosgene in which more than 5% of the phosgene prepared in the phosgene synthesis is reacted after the synthesis of the phosgene, usually from chlorine and carbon monoxide.

If one or more additionally, gaseous phosgene-free or amine-free inert streams are fed into the reaction space, these are regarded as a substream of the phosgene-comprising total stream in the calculation of the phosgene-comprising total stream when carrying out the process of the invention.

To carry out the process of the invention, it can be advantageous to preheat the streams of reactants, usually to temperatures of from 100 to 600° C., preferably from 200 to 450° C., before mixing.

In the amine stock vessel, the amine is preferably brought into the gas phase together with an inert medium as carrier gas, for example nitrogen, and fed into the mixing unit. However, the amine can also be vaporized directly without use of an inert medium. Phosgene is likewise brought into the gas phase, if appropriate together with an inert medium, from the phosgene stock vessel and introduced into the mixing unit.

In the process of the invention, the reactants are mixed in a mixing device in which the reaction stream passed through the mixing device is subjected to high shear. As mixing device, preference is given to a static mixing device or a mixing nozzle which is installed upstream of the reactor. Particular preference is given to using a mixing nozzle.

The type of mixing is immaterial according to the invention and mixing can be carried out in any way, for example as described in EP-B1 699657, EP-A2 1319655, column 1, line 54 to column 2, line 24 and column 4, lines 16-40, EP-A1 1275640, column 3, line 27-column 4, line 5, EP-A2 1362847, column 2, fine 19-column 3, line 51 and column 4, line 40-column 5, line 12, or the international patent application having the number PCT/EP2006/065593 and the filing date Aug. 23, 2006, p. 2, line 23 to p. 1, line 22, the full contents of which are in each case expressly incorporated by reference into the present disclosure.

According to the invention, phosgene is used in an excess over amino groups. The molar ratio of phosgene to amino groups is usually from 1.1:1 to 20:1, preferably from 1.2:1 to 5:1.

After mixing in the mixing unit, the gaseous mixture of phosgene, amine and, if appropriate, inert medium is fed into the reactor comprising the reaction space.

The reaction of phosgene with amine occurs in a reaction space which is generally located in a reactor, i.e. the reaction space is the space in which the major part of the reaction of the starting materials and intermediates occurs, for example at least 0.5 mol % of the amine used is converted into the corresponding isocyanate, preferably at least 1 mol %, particularly preferably at least 3 mol %, very particularly preferably at least 5 mol %, in particular at least 7 mol % and especially at least 10 mol %.

For the purposes of the present invention, the reactor is the industrial apparatus which comprises the reaction space. The reaction space can be any customary reaction space which is known from the prior art and is suitable for noncatalytic, single-phase gas reactions, preferably for continuous noncatalytic, single-phase gas reactions, and will withstand the moderate pressures required. Suitable materials for contact with the reaction mixture are, for example, metals such as steel, in particular alloy steel, tantalum, nickel, nickel alloys, silver or copper, glass, ceramic, enamels or homogeneous or heterogeneous mixtures and components composed of these. Preference is given to using steel apparatuses, particularly preferably steel reactors. The walls of the reactor can be smooth or profiled. Suitable profiles are, for example, grooves or corrugations.

In general, it is possible to use the reactor constructions known from the prior art. Examples of reactors are known from EP-B1 289840, column 3, line 49-column 4, line 25, EP-B1 593334, WO 2004/026813, p. 3, line 24-p. 6, line 10, WO 03/045900, p. 3, line 34-p. 6, line 15, EP-A1 1275639, column 4, line 17-column 5, line 17 and EP-B1 570799, column 2, line 1-column 3, line 42, the full contents of which are in each case expressly incorporated by reference into the present disclosure.

Preference is given to using tube reactors.

It is likewise possible to use essentially cuboidal reaction spaces, preferably plate reactors or plate reaction spaces. A particularly preferred plate rector has a ratio of width to height of at least 2:1, preferably at least 3:1, particularly preferably at least 5:1 and in particular at least 10:1. The upper limit to the ratio of width to height depends on the desired capacity of the reaction space and is in principle not limited. Reaction spaces having a ratio of width to height up to a maximum of 5000:1, preferably 1000:1, have been found to be industrially useful.

The reaction of phosgene with amine in the reaction space occurs at absolute pressures of from >0.1 bar to <20 bar, preferably from 0.5 bar to 15 bar and particularly preferably from 0.7 to 10 bar. In the case of the reaction of (cyclo)aliphatic amines, the absolute pressure is very particularly preferably from 0.7 bar to 5 bar, in particular from 0.8 to 3 bar and especially from 1 to 2 bar.

In general, the pressure in the feed lines to the mixing apparatus is higher than the abovementioned pressure in the reactor. The choice of mixing apparatus depends on this pressure. The pressure in the feed lines is preferably from 20 to 2000 mbar higher, particularly preferably from 30 to 1000 mbar higher, than in the reaction space.

In a preferred embodiment, the reactor comprises a bundle of reactors. In one possible embodiment, the mixing unit does not have to be a separate apparatus; however, it can be advantageous to integrate the mixing unit into the reactor. An example of an integrated unit made up of a mixing unit and reactor is a tube reactor having flanged-on nozzles.

In general, the pressure in the work-up apparatus is lower than in the reaction space. The pressure is preferably from 50 to 500 mbar lower, particularly preferably from 80 to 150 mbar lower, than in the reaction space.

In the process of the invention, the reaction of phosgene with amine occurs in the gas phase. For the purposes of the present invention, reaction in the gas phase means that the feed streams and intermediates react with one another in the gaseous state to form the products and over the course of the reaction during passage through the reaction space remain in the gas phase to an extent of at least 95%, preferably at least 98%, particularly preferably at least 99%, very particularly preferably at least 99.5%, in particular at least 99.8% and especially at least 99.9%.

Intermediates are, for example, the monoaminomonocarbamoyl chlorides, dicarbamoyl chlorides, monoaminomonoisocyanates and monoisocyanatomonocarbamoyl chlorides formed from the diamines and also the hydrochlorides of the amino compounds.

In the process of the invention, the temperature in the reaction space is chosen so that it is above the boiling point of the diamine used, based on the partial pressure conditions prevailing in the reaction space. Depending on the amine used and pressure set, an advantageous temperature in the reaction space is usually above 200° C., preferably above 260° C. and particularly preferably above 300° C. The temperature is generally up to 600° C., preferably up to 550° C.

The mean contact time of the reaction mixture in the process of the invention is generally in the range from 0.001 second to <5 seconds, preferably from >0.01 second to <3 seconds, particularly preferably from >0.015 second to <2 seconds. In the case of the reaction of (cyclo)aliphatic amines, the mean contact time can very particularly preferably be from 0.015 to 1.5 seconds, in particular from 0.015 to 0.5 second, especially from 0.020 to 0.1 second and often from 0.025 to 0.05 second.

In a preferred embodiment, the flow in the process of the invention has a Bodenstein number of more than 10, preferably more than 100 and particularly preferably more than 500.

In a preferred embodiment, the dimensions of the reaction space and the flow velocities are selected so that turbulent flow, i.e. flow having a Reynolds number of at least 2300, particularly preferably 2700, of the reaction mixture occurs, where the Reynolds number is formed using the hydraulic diameter of the reaction space.

The gaseous reaction mixture preferably passes through the reaction space at a flow velocity of from 10 to 300 meters/second, preferably from 25 to 250 meters/second, particularly preferably from 40 to 230 meters/second, very particularly preferably from 50 to 200 meters/second, in particular from >150 to 190 meters/second and especially from 160 to 180 meters/second. As a result of the turbulent flow, a narrow residence time having a small standard deviation of usually not more than 6% as described in EP 570799 and good mixing are achieved. Measures such as the constriction described in EP-A-593 334, which is also susceptible to blockages, are not necessary.

The reaction volume can be heated/cooled via its exterior surface. In order to build production plants having a high plant capacity, a plurality of reaction tubes can be connected in parallel. However, the reaction can also preferably be carried out adiabatically. This means that there are no heating or cooling energy flows resulting from engineering measures through the exterior surface of the reaction volume. The reaction preferably takes place adiabatically.

The process of the invention is preferably carried out in a single stage. For the purposes of the present invention, this means that the mixing and reaction of the starting materials occurs in one step and in one temperature range, preferably in the above-mentioned temperature range. Furthermore, the process of the invention is preferably carried out continuously.

After the reaction, the gaseous reaction mixture is preferably scrubbed with a solvent at temperatures above 130° C. (quench). Preferred solvents are hydrocarbons which may optionally be substituted by halogen atoms, for example chlorobenzene, dichlorobenzene and toluene. Particular preference is given to using monochlorobenzene as solvent. It is also possible to use the isocyanate as solvent. In the scrub, the isocyanate is selectively transferred into the scrubbing solution. The remaining gas and the scrubbing solution obtained are subsequently separated, preferably by means of rectification, into isocyanate, solvent, phosgene and hydrogen chloride. Preference is given to using the isocyanate.

After the reaction mixture has been reacted in the reaction space, it is passed to the work-up apparatus with quench. This is preferably a scrubbing tower in which the isocyanate formed is separated off from the gaseous mixture by condensation in an inert solvent while excess phosgene, hydrogen chloride and, if appropriate, the inert medium pass in gaseous form through the work-up apparatus. Preferred inert solvents are hydrocarbons which may optionally be substituted by halogen atoms, for example chlorobenzene, dichlorobenzene and toluene. The temperature of the inert solvent is preferably kept above the dissolution temperature of the carbamyl chloride derived from the amine in the quench medium selected. The temperature of the inert solvent is particularly preferably kept above the melting point of the carbamyl chloride derived from the amine.

The scrub can, for example, be carried out in a stirred vessel or in other conventional apparatuses, e.g. in a column or mixer-settler apparatus.

In process engineering terms, all extraction and scrubbing processes and apparatuses known per se can be used for a scrub in the process of the invention, e.g. those which are described in Ullmann's Encyclopedia of Industrial Chemistry, 6th ed, 1999 Electronic Release, Chapter: Liquid—Liquid Extraction—Apparatus. For example, these can be single-stage or multistage, preferably single-stage, extractions and also those operated in cocurrent or countercurrent, preferably countercurrent.

A suitable quench is known, for example, from EP-A1 1403248, column 2, line 39-column 3, line 18, of the European patent application having the number 06123629.5 and the filing date Nov. 7, 2006 or the European patent application having the number 06123621.2 and the filing date Nov. 7, 2006, which are expressly incorporated by reference into the present disclosure.

In this quench zone, the reaction mixture which consists essentially of the isocyanates, phosgene and hydrogen chloride and also chlorine and/or bromine is intensively mixed with the liquid sprayed in. Mixing is carried out so that the temperature of the reaction mixture is reduced by from 50 to 300° C., preferably from 100 to 250° C., from an initial 200-500° C. and the isocyanate comprised in the reaction mixture goes over completely or partly into the liquid droplets sprayed in as a result of condensation while the phosgene and the hydrogen chloride and also chlorine and/or bromine remain essentially completely in the gas phase.

The proportion of the isocyanate comprised in the gaseous reaction mixture which goes over into the liquid phase in the quench zone is preferably from 20 to 100% by weight, particularly preferably from 50 to 99.5% by weight and in particular from 70 to 99% by weight, based on the isocyanate comprised in the reaction mixture.

The reaction mixture preferably flows through the quench zone from the top downward. Below the quench zone, there is a collection vessel in which the liquid phase is precipitated, collected and removed from the reaction space via an outlet and is subsequently worked up. The gas phase which remains is removed from the reaction space via a second outlet and is likewise worked up.

The quench can, for example, be carried out as described in EP 1403248 A1 or as described in WO 2005/123665.

The liquid droplets are for this purpose produced by means of one- or two-fluid atomizer nozzles, preferably one-fluid atomizer nozzles, and, depending on the design, produce a spray cone angle from 10 to 140°, preferably from 10 to 120°, particularly preferably from 10° to 100°.

The liquid which is sprayed in via the atomizer nozzles has to have a good solvent capability for isocyanates. Preference is given to using organic solvents. In particular, aromatic solvents which may be substituted by halogen atoms are used. Examples of such liquids are toluene, benzene, nitrobenzene, anisole, chlorobenzene, dichlorobenzene (ortho, para), trichlorobenzene, xylene, hexane, diethyl isophthalate (DEIP), tetrahydrofuran (THF), dimethylformamide (DMF) and mixtures thereof, preferably monochlorobenzene.

In a particular embodiment of the process of the invention, the liquid sprayed in is a mixture of isocyanates, a mixture of isocyanates and solvents or isocyanate, with the quenching liquid used in each case being able to comprise proportions of low boilers such as HCl and phosgene. Preference is given to using the isocyanate which is prepared in the respective process. Since the reaction is stopped by the temperature reduction in the quench zone, secondary reactions with the isocyanates sprayed in can be ruled out. The advantage of this embodiment is, in particular, that the solvent does not have to be separated off.

In an alternative preferred embodiment, the inert medium which is used together with at least one of the starting materials and the solvent which is used in the quench are the same compound; very particular preference is given to using monochlorobenzene in this case.

Small amounts of by-products which remain in the isocyanate can be removed from the desired isocyanate by means of additional rectification, by stripping with an inert gas or crystallization, preferably by rectification.

In the subsequent optional purification step, the isocyanate is separated from the solvent, preferably by rectification. Residual impurities comprising, in particular, chlorine and/or bromine and also hydrogen chloride, inert medium and/or phosgene can likewise be separated off here, as described, for example, in DE-A1 10260092.

Streams which consist essentially of phosgene and/or hydrogen chloride gas but may also comprise proportions of chlorine are obtained from the quench and/or the purification stage. According to the invention, chlorine and/or bromine are separated off from the phosgene in at least part of these streams which comprise phosgene and/or hydrogen chloride gas and also chlorine and/or bromine and are recirculated to the reaction, so that the mass fraction of chlorine in the phosgene-comprising stream after any mixing with fresh phosgene and before mixing with the amine-comprising stream is less than 1000 ppm by weight and/or the mass fraction of bromine is less than 50 ppm by weight, as specified according to the invention.

In a conceivable embodiment, the phosgene-comprising gas stream has a chlorine content of at least 0.05 ppm by weight or even at least 0.1 ppm by weight, if not even at least 1 ppm by weight. At least 5 ppm by weight is also conceivable.

In a conceivable embodiment, the phosgene-comprising gas stream has a bromine content of at least 0.005 ppm by weight or even at least 0.01 ppm by weight, if not even at least 0.1 ppm by weight. At least 0.5 ppm by weight is also conceivable.

The separation of the mixture comprising hydrogen chloride and/or phosgene and/or solvent and/or chlorine and/or bromine is preferably effected by partial condensation and/or rectification and/or scrubbing. The separation is preferably carried out in a combination of a rectification and a scrub in any order.

### Scrub

Preferred scrubbing media are the solvents mentioned above as quenching media. Particular preference is given to using the same solvents as scrubbing medium and quench medium.

In a combined scrub and rectification, phosgene is scrubbed out from the HCl-comprising stream by scrubbing with a scrubbing medium, preferably toluene, chlorobenzene or dichlorobenzene, particularly preferably chlorobenzene. This produces a scrubbing medium laden with phosgene and hydrogen chloride and also generally chlorine. The separation of phosgene, hydrogen chloride (HCl) and molecular chlorine (Cl₂) from this laden scrubbing medium after the scrub is preferably carried out by distillation.

The scrub is operated at pressures of from 1 to 10 bar absolute, preferably from 1 to 5 bar absolute.

The scrub is preferably operated at temperatures of from −5 to −40° C., preferably from −15 to −35, particularly preferably from −20 to −30° C.

In process engineering terms, all absorption processes and apparatuses known per se can be used for such a scrub in the process of the invention, e.g. those described in Ullmann's Encyclopedia of Industrial Chemistry, 6th ed, 2000 Electronic Release, chapter: “Absorption” and there preferably in the subchapters “Design of Absorption Systems”, “Design of Absorption Equipment” and “Design of Desorption Equipment”. These can be, for example, single-stage or multistage, preferably multistage, absorptions and also those operated in cocurrent or countercurrent, preferably countercurrent.

Preference is given to using columns provided with valve trays, sieve trays, ordered packing or random packing and also pulsed columns or columns having rotating internals. Preference is given to the scrubbing liquid being finely dispersed by means of a nozzle and brought into contact with the gas phase.

According to the invention, the separation is carried out so as to give a phosgene stream which, if appropriate after mixing with fresh phosgene, has a chlorine content of less than 1000 ppm by weight and/or a bromine content of less than 50 ppm by weight.

For this purpose, the ratio of mixture to be separated to scrubbing liquid is set to from 5:1 to 0.5:1, preferably from 3:1 to 1:1 and particularly preferably from 2.5:1 to 1.5:1.

As a further embodiment, mention may be made of the use of ionic liquids as scrubbing liquid, as is described, for example, in WO 2006/029788, there particularly on p. 2, line 39 to page 11, line 25.

### Partial Condensation

As an alternative or in addition, the chlorine and/or bromine can be separated off from the stream comprising phosgene and hydrogen chloride by partial condensation using the process as has already been described in WO 2004/56758, there preferably on p. 11, line 14 to p. 13, line 16 and in the example. The disclosure is hereby expressly incorporated by reference.

This is preferably achieved by fractionation of a mixture comprising hydrogen chloride and phosgene and also chlorine and/or bromine, possibly solvents, low boilers and inerts in firstly at least one partial or total, preferably partial, condensation of the stream comprising phosgene and hydrogen chloride and also possibly solvents, then rectification or stripping in a column to remove hydrogen chloride and chlorine and/or bromine from the bottom product phosgene and subsequently preferably scrubbing of the overhead product hydrogen chloride with the process solvent to absorb the phosgene in the process solvent. To remove solvent residues and/or chlorine and/or bromine and/or hydrogen chloride from the phosgene, an after-purification by means of adsorption, for example on activated carbon, or by other suitable methods can subsequently be carried out.

The partial condensation can, if appropriate, be carried out in a plurality of stages and at various temperature and pressure levels; further rectification or stripping in a column to remove hydrogen chloride and/or chlorine and/or bromine from the condensed phosgene can subsequently be carried out.

The partial condensation of phosgene from the resulting mixture comprising hydrogen chloride, phosgene, chlorine and/or bromine and also possibly solvents and inerts is carried out in one or preferably more stages at temperatures of from −40° C., achievable by means of refrigerants, to 40° C., achievable by means of cooling water, depending on the pressure in the reaction section.

### Rectification

The rectification for removing chlorine and/or bromine and if appropriate hydrogen chloride from the condensed phosgene obtained in this way is carried out at a temperature at the bottom of from 5 to 150° C., preferably from 5 to 50° C., a pressure at the top of from 1 to 35 bar, preferably from 1.5 to 4.0 bar, and a temperature at the top of from −20° C. to 30° C., preferably from −10° C. to 0° C.

The rectification can be carried out in commercial distillation columns having, for example, from 3 to 30, preferably from 5 to 25, theoretical plates.

As an alternative, the chlorine and/or bromine and, if appropriate, the hydrogen chloride can also be removed from the recycle phosgene by stripping with an inert gas such as nitrogen, the process solvent vapor, phosgene or another gaseous substance or substance to be vaporized.

### Adsorption on Activated Carbon

The phosgene stream coming from a phosgene production plant or obtained after distillation of the mixture comprising phosgene and hydrogen chloride can already have a chlorine content which is so low that it meets the criteria of the invention, but it is also possible for a further after-treatment to be necessary.

Such an after-treatment can preferably be effected by absorption of the chlorine and/or bromine comprising the phosgene on activated carbon, as is described, for example, in U.S. Pat. No. 3,331,873.

Activated carbon can adsorb considerable amounts of chlorine and/or bromine, for example up to 20% by weight, preferably up to 16% by weight, and can be regenerated by heating to temperatures above 70° C., preferably 150-250° C.

The activated carbon which can be used in the process can be any commercial activated carbon. The mode of action depends partly on a large ratio of its surface area to its mass. Activated carbons are obtainable in various degrees of porosity of the individual particles. Both activated carbons from minerals and activated carbons obtained from animal or vegetable sources are suitable for the process. An activated carbon having a relatively small pore radius, for example from about 2 to 3.5 Å, preferably from 2 to 3 Å and particularly preferably about 2.5 Å, has been found to be particularly effective and is therefore preferred.

The activated carbon can be present as any shaped bodies, for example as rods, powder, pellets, granules, compacts or extrudate.

Preference is given to a filled tower or a filled column as are usually employed for separating components from mixtures by means of activated carbon. Other apparatuses such as vessels having filter layers and equipped with powerful and fast-running stirrers can also be used. Facilities for heating or cooling the activated carbon are preferably provided. The process itself can be carried out batchwise or preferably continuously.

In an example of an embodiment of the process, a chlorine- and/or bromine-comprising phosgene stream is passed through an absorption tower which is charged with activated carbon and surrounded by a heat exchanger through which cooling brine circulates continuously. The crude phosgene enters the column, which has been cooled to a temperature below 8° C., preferably from 0 to −10° C., and is maintained at this temperature, at its lower end and flows through the activated carbon. The purified phosgene leaves the column at its upper end or in the vicinity thereof.

The exhausted activated carbon can be regenerated by heating it to a temperature of at least 70° C., preferably 150-250° C.

To carry out the regeneration, a stream of a gas which is inert under the regeneration conditions, for example nitrogen, argon, carbon dioxide or preferably carbon monoxide, can be passed through the activated carbon at a flow rate of 120-370 kg/m² of cross section of the activated carbon bed and hour until it is virtually free of phosgene, i.e. comprising less than 0.05% by volume of phosgene.

According to the invention, the separation of phosgene, solvent from the quench, i.e. preferably monochlorobenzene, hydrogen chloride and chlorine is preferably carried out in the following preferred combinations of process steps, in particular a combination of partial condensation and scrubbing or a combination of partial condensation and rectification.

The mixture to be separated generally has the following composition:
solvent from quench 2-60% by weight, preferably from 5 to 40% by weight, particularly preferably 10-30% by weight,
phosgene 20-95% by weight, preferably 40-85% by weight, particularly preferably 60-75% by weight,
hydrogen chloride 1-50% by weight, preferably 2-30% by weight, particularly preferably 5-20% by weight and very particularly preferably 5-15% by weight,
chlorine 10-10 000 ppm by weight, preferably 100-5000 ppm by weight, particularly preferably 250-3000 ppm by weight and very particularly preferably 500-2000 ppm by weight,
bromine 0.01-100 ppm by weight, preferably 0.05-50 ppm by weight, particularly preferably 0.1-10 ppm by weight and very particularly preferably 0.2-10 ppm by weight, and
diisocyanate, its intermediates and subsequent products (total) 0-10% by weight, preferably 0-5% by weight, particularly preferably 10 ppm by weight-3% by weight and very particularly preferably from 100 ppm by weight to 1% by weight, with the proviso that the sum is 100% by weight.

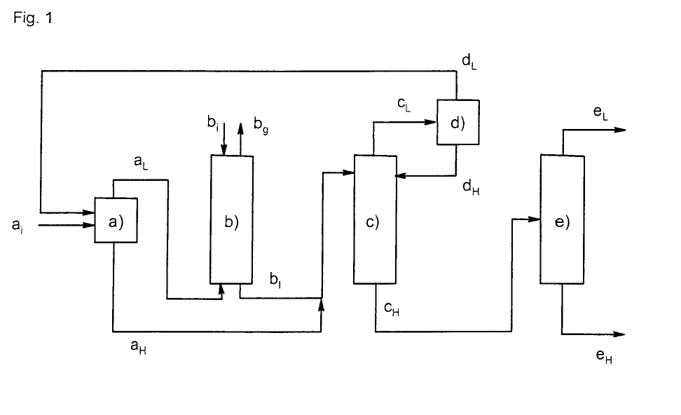

In a first, preferred embodiment, as shown in FIG. 1, this mixture to be separated aᵢ is introduced into a partial condensation in step a) giving a condensate a_{H} which comprises predominantly solvent, reduced amounts of phosgene and traces of hydrogen chloride and chlorine and a gaseous stream a_{L} which consists predominantly of phosgene, hydrogen chloride and chlorine together with minor amounts of solvent. This partial condensation is generally operated at a pressure of from 0.1 to 20 bar abs, preferably from 0.2 to 10 bar abs., particularly preferably from 0.5 to 5 bar abs. and very particularly preferably from 0.8 to 2 bar abs. The stream leaving the condensation has, for example, a temperature of from −5 to −40° C., preferably −15 to −35° C., particularly preferably −20 to −30° C.

The gaseous stream a_{L} obtained in this way is then fed to a scrub b) in which this stream is treated, preferably in countercurrent, with a scrubbing liquid bᵢ, as described above. Scrubbing liquid and solvent in the mixture to be separated are preferably identical. The scrub b) gives a gaseous stream b_{g} from which phosgene and solvent have been virtually completely removed and which consists essentially of hydrogen chloride, chlorine and/or bromine. Scrubbing liquid can also be comprised, for example as a result of entrainment. Liquid constituents of the stream b_{g} can be removed by means of downstream dephlegmators or droplet precipitators. The stream b_{g} can then be passed, if appropriate, after further purification steps, to processes for utilizing hydrogen chloride, for example as described in DE 10235476 (corresponds to U.S. Pat. No. 6,916,953).

Such a utilization of hydrogen chloride can preferably be chlorine production, particularly preferably an electrolysis or very particularly preferably a Deacon process.

The liquid output b_{I} from the scrubbing stage b) then consists predominantly of scrubbing liquid in which the major part of the phosgene from the stream a_{L} introduced into stage b) is present. In addition, small amounts of hydrogen chloride and traces of chlorine can also be present in the scrubbing liquid.

The scrub is preferably configured so that it has from 2 to 25 thermodynamic stages, particularly preferably from 5 to 20 thermodynamic stages and very particularly preferably from 10 to 15 thermodynamic stages.

The scrub is generally operated at a pressure of from 0.1 to 20 bar abs, preferably from 0.2 to 10 bar abs., particularly preferably from 0.5 to 5 bar abs. and very particularly preferably from 0.8 to 2 bar abs. The temperature of the scrubbing medium is, for example, from −5 to −40° C., preferably from −15 to −35° C., particularly preferably from −20 to −30° C.

The ratio of reaction mixture to scrub liquid is, for example, from 5:1 to 0.5:1, preferably from 3:1 to 1:1 and particularly preferably from 2.5:1 to 1.5:1.

The streams b_{I} and a_{H} are then combined and fed to a rectification c) in which essentially the low boilers chlorine and hydrogen chloride are separated off from phosgene and scrubbing liquid. In a preferred embodiment, the rectification c) comprises only a stripping section, i.e. the feed stream to the rectification c) is fed into the rectification above the separation-active internals. The stripping section generally comprises from 5 to 30 theoretical plates, preferably from 10 to 25 theoretical plates and particularly preferably from 15 to 20 theoretical plates. In a less preferred embodiment, the rectification c) can additionally comprise an enrichment section having from 2 to 10 theoretical plates. The rectification c) is generally operated at a temperature at the top of from 0 to 25° C., preferably from 5 to 20° C. and particularly preferably from 10 to 15° C., a temperature at the bottom of from 20 to 80° C., preferably from 30 to 70° C. and particularly preferably from 40 to 60° C., and a pressure of from 1 to 5 bar abs, preferably from 2 to 3.5 bar abs.

The rectification can, for example, be carried out at a reflux ratio of from 1 to 20, preferably from 3 to 15 and particularly preferably from 5 to 12.

The high boiler stream c_{H} consists essentially of phosgene and scrubbing liquid. The vapor stream c_{L} can comprise not only hydrogen chloride and chlorine but also small amounts of phosgene and scrubbing liquid.

In a preferred embodiment, the vapor stream c_{L} is partially condensed in a stage d), and the condensate d_{H} is recirculated to stage c). The gaseous stream d_{L}, which comprises predominantly hydrogen chloride and chlorine together with a little phosgene, is then introduced into stage a).

If the fresh phosgene fed into the overall process has a high chlorine content, for example above 2 ppm by weight, preferably above 10 ppm by weight, particularly preferably above 20 ppm by weight, very particularly preferably above 50 ppm by weight and in particular above 100 ppm by weight, which is to be separated off in the process of the invention, this chlorine-comprising fresh phosgene is, according to the invention, passed at least in part, preferably in its entirety, over separation-active internals of a rectification column before being reacted with the amine. This can be achieved, for example, by this chlorine-comprising fresh phosgene preferably being fed to the stage c), particularly preferably at the same theoretical plate as the other streams which are fed into stage c). This can be effected, for example, by mixing with the streams b_{I} and a_{H} and subsequent introduction into stage c).

This rectification column is preferably a constituent of the separation of the mixture comprising hydrogen chloride and phosgene and possibly inert medium from the reaction stage of the gas-phase phosgenation.

The stream c_{H} is then fed into a last rectification e) in which phosgene and the scrubbing liquid are separated from one another. The stage e) should preferably have at least a stripping section having from 2 to 20 theoretical plates, preferably from 5 to 15 theoretical plates, particularly preferably from 6 to 10 theoretical plates, so that the high boiler stream e_{H} obtained at the bottom consists essentially of pure scrubbing liquid and the low boiler stream e_{L} is taken off without rectification over separation-active internals in the top of the rectification e). In this case, the stream e_{H} consists essentially of scrubbing liquid and phosgene and can be recirculated, preferably without further purification, to the phosgenation, since the scrubbing liquid then acts as inert medium in the gas-phase phosgenation.

In a particularly preferred embodiment, stage e) has both an enrichment section having preferably from 0.5 to 10 theoretical plates, preferably from 1 to 5 theoretical plates, and a stripping section having from 2 to 20 theoretical plates, preferably from 5 to 15 theoretical plates and particularly preferably 6-10 theoretical plates, and is operated at a temperature at the top of from 5 to 80° C., preferably from 10 to 60° C. and particularly preferably from 20 to 40° C., a temperature at the bottom of from 100 to 200° C., preferably from 130 to 180° C. and particularly preferably from 150 to 175° C., and a pressure of from 1 to 5 bar abs., preferably 2 to 3.5 bar abs.

The rectification of stage e) is, for example, operated at a reflux ratio of from 0.1 to 10, preferably from 0.2 to 5 and particularly preferably from 0.5 to 2.

The vapor stream e_{L} obtained here consists essentially of pure phosgene which can then be used, preferably without further purification, in the phosgenation. The high boiler stream e_{H} consists essentially of pure scrubbing liquid which can then be used, preferably without further purification, in the scrub b) and/or the quench.

In a second embodiment, all steps of the first embodiment are implemented, with the difference that the distillation stages c) and e) are combined in a dividing wall column. This dividing wall column is preferably configured so that the inflow side has only a stripping section and the outflow side has exclusively an enrichment section. The dividing wall then separates the gas spaces of inflow side and outflow side from one another.

The gas space of the inflow side is then once again preferably connected to a partial condensation d). In such an embodiment, the stream c_{L} is taken off from the gas space of the inflow side, the stream e_{L}, which consists essentially of phosgene, is taken off from the gas space of the outflow side and the stream e_{H}, which consists essentially of the scrubbing liquid, is taken off from the combined bottom of outflow and inflow side.

In a third alternative embodiment, all features of the first embodiment are once again implemented and the distillation stages c) and e) are likewise combined in a dividing wall column. However, in this case this dividing wall column is preferably configured so that both the inflow side and the outflow side each have a stripping section and an enrichment section. Low boiler space and high boiler space are then connected to one another via separation-active internals. The dividing wall separates inflow side and outflow side from one another.

The combined gas space is once again preferably connected to a partial condensation d). In such an embodiment, the stream CL is taken off from the combined gas space, the stream e_{L}, which consists essentially of phosgene, is now taken off from the outflow side as intermediate boiler and the stream e_{H}, which consists essentially of the scrubbing liquid, is taken off from the combined bottom of outflow and inflow side.

According to the invention, preference is given to those combinations of process steps for separating mixtures comprising phosgene, solvent from the quench or scrubbing liquid from step b), hydrogen chloride and chlorine in which the scrubbing liquid has been purified at least once by rectification over at least one theoretical plate before it is recirculated to the scrub b) and/or the quench.

If the fresh phosgene fed to the overall process has a high chlorine content and/or bromine content, i.e. above the limits indicated above, which is to be separated off in the process of the invention, preference is also given, according to the invention, to those combinations of process steps in which the phosgene has been purified at least once by rectification over at least one theoretical plate before it is fed to the phosgenation.

The low content of chlorine and/or bromine specified according to the invention can preferably be achieved as follows:

1) The fresh phosgene fed to the process has the required low chlorine and/or bromine content.

This is particularly preferred for achieving the low bromine content according to the invention.

The low chlorine and bromine contents can be achieved by treatment of the fresh phosgene fed to the process of the invention, for example with activated carbon, or by means of a specific procedure in the synthesis of the phosgene from chlorine and carbon monoxide.

Phosgene is frequently prepared by bringing carbon monoxide into contact with molecular chlorine (Cl₂) over suitable supports, preferably activated carbon. Since the formation of phosgene is strongly exothermic, this is preferably carried out in shell-and-tube reactors in which the bed of support is heated to temperatures of up to 400° C. by the exothermic reaction, but the temperature drops to 40-150° C. during passage through the tubes. The heat of reaction liberated is removed by means of suitable heat transfer media, for example water. The pressure is usually atmospheric pressure or slightly above atmospheric pressure.

If the preparation of phosgene is carried out using a stoichiometric excess of carbon monoxide and a sufficiently long residence time, complete reaction of the chlorine is generally achieved. The phosgene obtained in this way still comprises small amounts of carbon monoxide. However, this does not have any adverse effect when the phosgene is employed in phosgenation, since the carbon monoxide comprised functions as inert gas in the gas-phase phosgenation.

2) If the fresh phosgene has a relatively high chlorine content, for example above 2 ppm by weight, preferably above 10 ppm by weight, particularly preferably above 20 ppm by weight, very particularly preferably above 50 ppm by weight and in particular above 100 ppm by weight, the fresh phosgene cannot, according to the invention, be introduced directly into the reaction with the amine, but is introduced at least in part, preferably in its entirety, into the separation of the mixture comprising phosgene and hydrogen chloride obtained from the phosgenation, where residues of chlorine and/or bromine are separated off from the phosgene together with hydrogen chloride. This represents a preferred embodiment of the present invention.

This is advantageous since the chlorine formed by dissociation of phosgene or coming from the preparation of the phosgene can in this way be separated off before the phosgenation.

The hydrogen chloride/phosgene separation can be followed by an adsorption unit, preferably an activated carbon filter, in the hydrogen chloride stream discharged from the separation so as to remove traces of the scrubbing medium from the hydrogen chloride obtained.

The diisocyanates prepared by the gas-phase phosgenation process are highly suitable for preparing polyisocyanates for use in polymers comprising urethane, isocyanurate, amide and/or urea groups produced by the polyisocyanate polyaddition process. They are also used for preparing polyisocyanate mixtures modified with urethane, biuret and/or isocyanurate groups. Such polyisocyanate mixtures comprising aliphatic or cycloaliphatic diisocyanates are used, in particular, for producing light-stable polyurethane paints, varnishes and coatings and also in thermoplastic polyurethanes.

1. A process for preparing diisocyanates by reacting the corresponding diamines with a stoichiometric excess of phosgene in at least one reaction zone,
wherein the reaction conditions are selected so that at least the reaction components diamine, diisocyanate and phosgene are gaseous under these conditions and
at least one diamine-containing gas stream and at least one phosgene-containing gas stream are fed into the reaction zone,
with the mass fraction of chlorine in the phosgene-containing stream before mixing with the amine-containing stream being less than 1000 ppm by weight and/or the mass fraction of bromine in the phosgene-containing stream being less than 50 ppm by weight.

2. The process according to claim 1, wherein at least one inert medium is added to the diamine-containing and/or phosgene-containing gas stream so that the gas volume ratio of inert medium to amine or to phosgene is from >0.0001 to 30.

3. The process according to claim 1, wherein the reaction of phosgene with diamine in the reaction space is carried out at absolute pressures of from >0.1 bar to <20 bar.

4. The process according to claim 1, wherein the reaction of phosgene with diamine in the reaction space is carried out at temperatures of from >200° C. to 600° C.

5. The process according to claim 1, wherein the mean contact time of the reaction mixture is from 0.001 second to <5 seconds.

6. The process according to claim 1, wherein the molar ratio of phosgene to amino groups is from 1.1:1 to 20:1.

7. The process according to claim 1, wherein the flow in the reaction space has a Bodenstein number of more than 10.

8. The process according to claim 1, wherein the mass fraction of hydrogen chloride in the phosgene-comprising stream after any mixing with fresh phosgene and before mixing with the amine-comprising stream is less than 15% by weight.

9. The process according to claim 2, wherein the inert medium is chlorobenzene.

10. The process according to claim 1, wherein the isocyanate is selected from the group consisting of 1,6-diisocyanatohexane, 1-isocyanato-3,3,5-trimethyl-5-(isocyanatomethyl)cyclohexane, 4,4′-di(isocyanatocyclohexyl)methane and tolylene 2,4-/2,6-diisocyanate isomer mixtures.

11. The process according to claim 1, wherein fresh phosgene having a proportion of molecular chlorine (Cl₂) above 2 ppm by weight is at least partly passed over separation-active internals of a rectification column to separate the chlorine from the phosgene before it is reacted with the amine.

12. The process according to claim 11, wherein the rectification column is a constituent of the separation of a mixture comprising hydrogen chloride and phosgene and possibly an inert medium.

13. A gas phase phosgenation process wherein phosgene having a content of molecular chlorine (Cl₂) of less than 1000 ppm by weight is fed to the reaction zone.

14. A gas-phase phosgenation process wherein phosgene having a content of molecular bromine (Br₂) of less than 50 ppm by weight is fed to the reaction zone.
